(19) United States
(12) Patent Application Publication
(10) Pub. No.: US 2010/0196441 A1
(43) Pub. Date: Aug. 5, 2010

(54) USES OF IMMUNOLOGICALLY MODIFIED SCAFFOLD FOR TISSUE PREVASCULARIZATION CELL TRANSPLANTATION

(75) Inventors: Hugo P. SONDERMEIJER, New York, NY (US); Piotr WITKOWSKI, New York, NY (US); Mark A. HARDY, Scarsdale, NY (US)

Correspondence Address: LAW OFFICES OF ALBERT WAI-KIT CHAN, PLLC, 141-07 20th AVENUE, WORLD PLAZA, SUITE 604, WHITESTONE, NY, 11357, US

(73) Assignee: The Trustees of Columbia University in the City of New York, New York, NY (US)

(21) Appl. No.: 12/699,426

(22) Filed: Feb. 3, 2010

Related U.S. Application Data
(63) Continuation-in-part of application No. PCT/US2008/076695, filed on Sep. 17, 2008.
(60) Provisional application No. 61/050,667, filed on May 6, 2008.
(60) Provisional application No. 60/973,074, filed on Sep. 17, 2007.

(51) Int. Cl. A61F 2/24; A61K 35/12; A61K 35/28; A61K 35/55; A61K 35/34; A61K 38/18; A61K 38/17; A61K 39/395; A61K 38/02; A61P 9/00; C12N 5/00

(52) U.S. Cl. 424/426; 424/93.7; 514/8; 514/12; 424/130.1; 514/2; 424/93.1; 435/395

(57) ABSTRACT

This invention provides method of making and using of a porous 3 dimensional cyclic RGD peptide-modified alginate scaffold that can be loaded with different cell types and/or growth factors for implantation at sites of tissue damage to promote tissue regeneration. The cyclic RGD peptide promotes vascular formation of the host tissue, cell binding and survival of seeded cells. Scaffolds with growth factors but without cells can also be implanted to create a vascular bed in which cells are transplanted at a later time point.

### CROSS-REFERENCE TO RELATED APPLICATIONS

This is a continuation-in-part application of International Application No. PCT/US2008/076695, filed Sep. 17, 2008, which claims benefit of U.S. Application No. 61/050,667, filed May 6, 2008, and U.S. Application No. 60/973,074, filed Sep. 17, 2007. The entire contents and disclosures of the preceding applications are incorporated by reference into this application.

### STATEMENT REGARDING FEDERALLY SPONSORED RESEARCH

This invention was funded in part by a grant of the U.S. federal government, NIH SCCOR # 5P50HL077096-02. The government has certain rights in the invention.

### BACKGROUND OF THE INVENTION

New treatment modalities for cardiovascular diseases are needed and cell therapy is a promising new option. Cells can be directly injected into damaged heart tissue to generate new vessels and salvage myocardium. Unfortunately, clinical trials using catheter based cell injection into myocardium following acute myocardial infarction (MI), which is the current standard delivery method, have demonstrated only modest beneficial effects on cardiac function. This result may be explained by the fate of transplanted cells, which is currently elusive.

In order to address these problems, various novel approaches have been employed, for example by delivering cells in collagen or fibrin biomaterial carriers (Christman and Lee, 2006). These carriers provide survival signaling, mediate cell adhesion and promote neoangiogenesis through their RGD (Arg-Gly-Asp) amino acid sequences which interact with integrin receptors on the cell surface, and have been shown to promote cell survival after intramyocardial injection (Christman et al., 2004). A disadvantage of these carriers is their intrinsic property to induce unwanted immune responses and the presence of animal derived components which limited their clinical application.

Alginate, a natural, biodegradable polysaccharide derived from seaweed, has several distinct advantages over the aforementioned biomaterials. It is non-toxic and non-animal derived and therefore eliminates the risk of viral or prion contamination. It is also cheap and readily available, making it attractive for large scale clinical applications. Raw, unpurified alginate contains contaminating factors that can induce a host immune response. However, when thoroughly purified, it has no significant immunogenic properties (Zimmermann et al., 2001). It can be modified by covalent binding with RGD or other bioactive peptides, which benefits cell survival, cell adhesion and angiogenesis.

Transplantation of cells to the infarcted heart using 3-dimensional scaffolds or sheets has previously been shown to improve cardiac remodeling and induce cardiac regeneration (Leor et al., 2000; Miyahara et al., 2006). Leor et al. used unmodified alginate scaffolds to transplant cells to the infarcted myocardium. They found that scaffold transplantation without cells produced a similar beneficial effect on cardiac remodeling as did cell containing scaffolds. This may be explained by a lack of survival and/or retention of cells inside the unmodified scaffold, since unmodified alginate does not interact with mammalian cells. Hill et al. showed that cell seeded alginate scaffolds enriched with RGD peptides and growth factors augmented muscle regeneration in a hind limb muscle injury model more effectively than non-enriched scaffolds (Hill et al., 2006a). Therefore, it is believed that the modification of alginate with adhesion and survival factors will improve cell retention and cell survival, and add to the beneficial effects of alginate scaffold transplantation on infarcted myocardium.

Commercially available “ultrapure” alginate preparations still contain significant amounts of contaminating material such as proteins which could induce unwanted host immune responses (Dusseault et al., 2006). Thus, there is a need to develop a novel method to generate highly purified alginate and to demonstrate that alginate scaffolds fabricated from this material would induce scaffold angiogenesis after enrichment with survival factors such as protease-resistant cyclic RGD peptides, PDGFbb and VEGF.

### SUMMARY OF THE INVENTION

This invention describes the purification of commercially available unpurified alginate and subsequent fabrication of tissue engineered alginate scaffolds for tissue prevascularization, cell transplantation and tissue regeneration. Purification of alginate is based on a customized process that removes virtually all contamination with protein, DNA, RNA and endotoxin. In one embodiment, fabrication comprises cyclic RGD peptide conjugation to purified liquid alginate using carbodiimide chemistry followed by scaffold generation using alginate solidification by divalent ions, for example, Ca²⁺ or Ba²⁺. Solid scaffolds can be generated using a transwell system; porous scaffolds can be generated by freeze gelation. Scaffold may be implanted together with seeded cells and/or modulating factors days/weeks before cells transplantation which permits proper preconditioning of the transplant “bed” including prevascularization and/or immunomodulation, leading to improved cell engraftment and survival. In another embodiment, modified alginate may be injected in combination with cells and/or growth factors directly into tissue in order to provide cell survival and retention.

### BRIEF DESCRIPTION OF THE DRAWINGS

FIG. 1 shows scaffold generation by freeze gelation (“dry scaffold”). Alginate solution is cast in a silicone mold punched out in the middle sheet of a 3 silicone sheet sandwich. After layering, the sandwiched sheets+alginate are frozen at −20° Celsius. After freezing, resulting solid alginate disc is placed in 1.1% calcium chloride in 70% ethanol/ddH₂O solution at −20° Celsius for 24 h. After solidification, solid disc is washed 3× in ddH₂O, followed by 3× wash in 100% ethanol, followed by air drying. At least 24 hours of drying before adding cells, and/or soluble factors.

FIG. 2 shows 3D RGD-alginate dry scaffold fabrication. Custom purified 3D alginate scaffold generation using a combination of freeze gelation and ethanol evaporation resulted in highly porous material with pore sizes between 25 μm-100 μm.

FIG. 3 shows scaffold generation using transwell system (“wet scaffold”). Alginate solution is cast in a transwell containing semi-permeable membrane. Transwell is placed in bottom well containing 1.1% calcium solution. After 24 hours, the alginate is solidified and removed from the transwell. Soluble factors can be added to the alginate solution before solidification in order to generate a sustained release alginate disc.

FIG. 4 shows effect of cRGDfk peptide on cell proliferation and neovascularization. Dry non-modified and cRGDfK modified (20 mg cRGDfK per gram alginate) scaffolds were implanted between abdominal muscles of immunocompetent rats. Thirty days after implantation, scaffolds were harvested and assessed for cell infiltration and neovascularization. Non-modified scaffolds showed minimal cell infiltration, whereas cRGDfK modified scaffolds showed abundant cellular ingrowth and scaffold vascularization. No evidence of inflammation was detected.

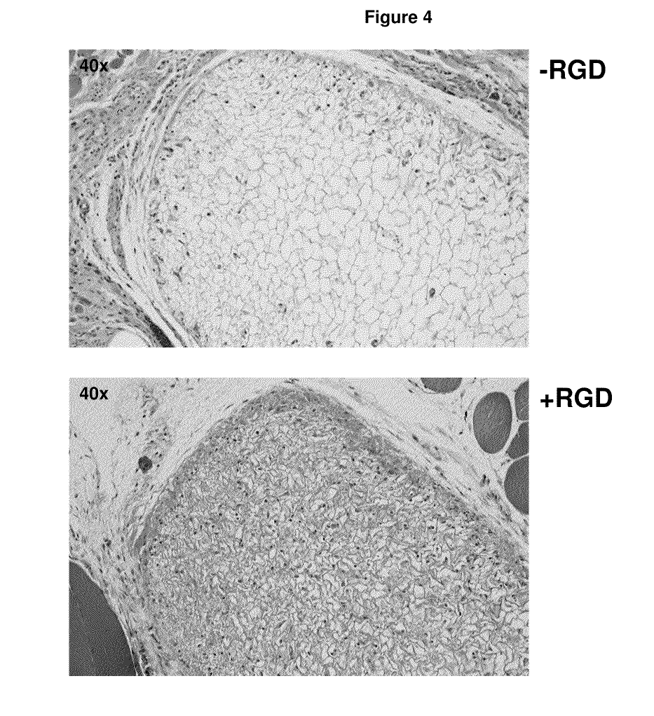

FIG. 5 shows effect of addition of PDGFbb and VEGF to cRGDfK scaffold. cRGFfK scaffolds were impregnated with 100 ng/ml PDGFbb and 100 ng/ml VEGF. Vessel formation was determined by alpha smooth muscle actin staining. Addition of PDGFbb and VEGF resulted in significant increase of neovascularization around and throughout the scaffold (shown at arrows).

FIG. 6 shows histology of epicardial scaffold application. cRGDfK scaffolds (20 mg cRGDfK per gram alginate) seeded with human mesenchymal precursor cells were applied to the epicardium 2 days after myocardial infarction and harvested for histology after 1 week. Staining was done for endothelial cells (fVIII). Scaffolds can be identified on the epicardium (labeled S). Vascular formation was most evident in the border zones of the infarcted heart (arrows).

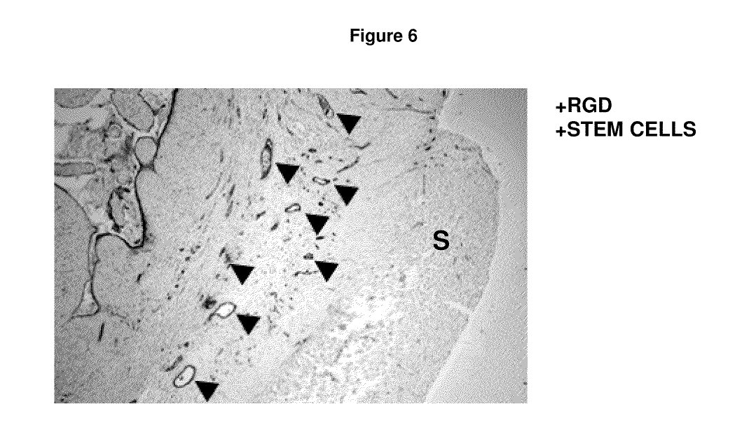

FIG. 7 shows left ventricular wall 1 week following infarction. Masson's trichrome staining for fibrosis on the top panel showed scaffold (Scaf) cellularization with minimal fibrosis (blue). Bottom panel shows ED-2 staining for macrophages. There was no evidence of foreign body reaction against the material at 1 week following implantation. Inf=infarct. LV=left ventricle.

FIG. 8 shows cardiac function after epicardial scaffold application. Fractional shortening by echocardiography showed significant increase in cardiac function 1 week following epicardial application of scaffolds seeded with 1 million hMSCs. This effect was not observed using control scaffolds or scaffolds seeded with 3 million hMSCs. *p≦0.05

FIG. 9 shows numbers of erythrocyte filled blood vessels in infarct zone, border zone and scaffold after epicardial scaffold application. Border zone vessel numbers significantly increased using scaffolds with 1 million hMSCs. *p≦0.05.

FIG. 10 shows scaffold imaging using positron emission tomography (PET). An image of an animal which fully controlled glycemia after islet transplantation into scaffold with PDGFbb and VEGF was shown with high activity area that corresponded to transplant islet site (arrow). No activity was observed in sham-operated animals with primary non-functional of islets.

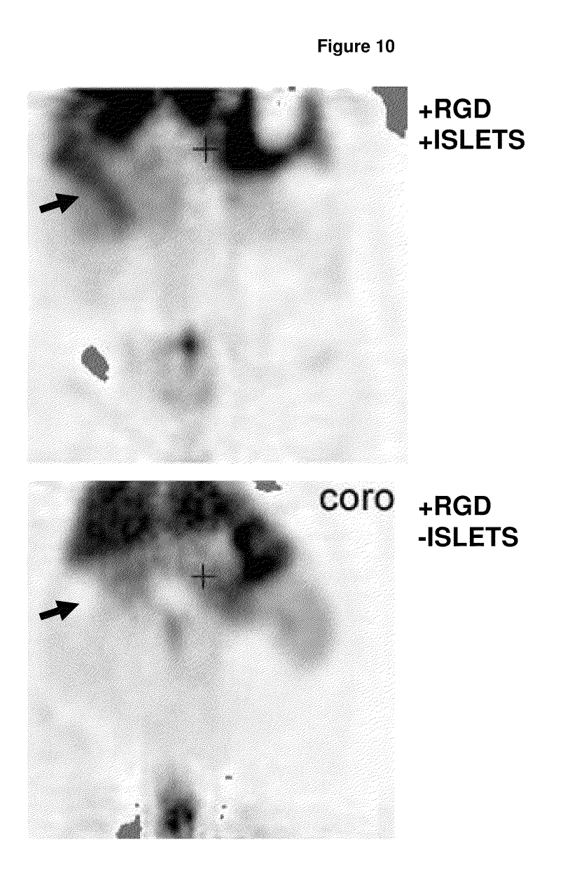

FIG. 11 shows insulin staining after scaffold+islet implantation. Sixty days after implantation, removed tissue stained for insulin was presented. Cells staining positively for insulin were seen within the scaffold, especially in proximity of vessels at the scaffold-muscle interface.

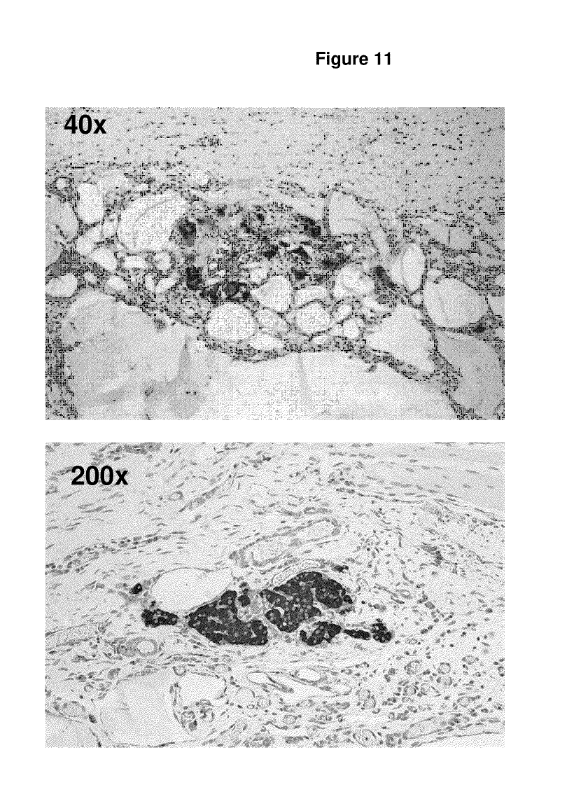

### DETAILED DESCRIPTION OF THE INVENTION

The following terms shall be used to describe the present invention. In the absence of a specific definition set forth herein, the terms used to describe the present invention shall be given their common meaning as understood by those of ordinary skill in the art.

As used herein, “cyclic RGD peptides” refer to synthetic peptides comprising an RGD amino acid sequence and additional amino acids to establish cyclicalisation. In general, the cyclic RGD peptides are cyclo RGDxy, where “x” can be D-phenylalanine or D-tyrosine which binds to the “R” residue, and “y” can be L-cysteine, L-glutamic acid, L-lysine or L-valine for further linker functions. In one embodiment, the cyclic peptide is cyclo RGDfK, where f=D-phenylalanine and K is L-lysine. In another embodiment, the cyclic peptide comprises GPenRGDSPCA, wherein “Pen2” (penicillamine) binds to “C9” through cysteine bonds.

As used herein, “dry scaffold” refers to 3-dimensional scaffolds as described in paragraphs [0027]-[0029].

As used herein, “wet scaffold” refers to 3-dimensional scaffolds as described in paragraphs [0030] and [0031].

Alginic acid, also called algin or alginate, is an anionic polysaccharide distributed widely in the cell walls of brown algae. It is a linear co-polymer of mannuronic acid and guluronic acid, the relative amounts of which vary greatly between alginic acids from different species of algae. Additionally, alginic acids from different sources vary in the arrangement of the uronic acids within the molecule so that alginic acid may be considered as a co-polymer consisting of homopolymeric blocks of mannuronic acid and of guluronic acid. Commercial varieties of alginate are extracted from seaweed, for example the giant kelp Macrocystis pyrifera, Ascophyllum nodosum, and various types of Laminaria. It is also produced by two bacterial genera Pseudomonas and Azotobacter.

### Alginate Purification

In one embodiment, 1.5% (weigth/weight) low molecular weight alginate (Sigma-Aldrich 0682) composed primarily of 1,4-poly-mannuronic acid was dissolved in 1000 ml 10 mM sodium phosphate buffer, pH 5.5 at 20° C. Buffer was composed of 1.32 grams per liter monosodium phosphate monohydrate and 0.11 grams per liter disodium phosphate heptahydrate in ddH₂O. The solution was stirred at room temperature until alginate was dissolved. 1.5% neutral carbon was added and pH was adjusted to 5.5 using 37% HCl and stirred at 50° C. for 24 hours. Then, the solution was filtered through a glass prefilter, treated with 1.5% active carbon at pH 5.5, stirred at 50° C. for 24 h and filtered through glass prefilter. Afterwards, the solution was kept at 4° C. for 24 hours. Subsequently, the solution was filtered through hydrophobic Immobilon P membranes at room temperature, pH 5.5, 50 ml per 90 mm filter in a Buchner funnel. The solution was then dialyzed using 50000 MWCO tubing for 48 h against ddH₂O, frozen at minus 20° C. and lyophilized. After lyophilization, the product was dissolved at 2% (weight/weight) in endotoxin free H₂O, Subsequently, ethanol 200 proof at a ratio of 1:1 was added and the solution was vortexed at 3000 rpm for 1 minute. The solution was spun at 4000 rpm for 30 minutes at 4° C. Supernatant was removed and pellets were frozen at minus 80° C. and subsequently freeze-dried at 0.1 mm Hg. Resulting product was then redissolved at 2% (weight/weight). Pierce Micro BCA assay was used to determine the presence of protein. Qubit was used to determine DNA or RNA contamination. Endotoxin presence was determined by using the Pyrosate kit (Cape Cod).

### RGD Alginate Coupling

In one embodiment, using carbodiimide chemistry (EDC and sulfo-NHS in MES buffer, Pierce), 1-20 mg cyclic RGD peptides cRGDxy (xy being any combination of amino acids) (American Peptide Co.), cGPenGRGDSPCA (Peptides International) or other cyclic peptides as described above can be covalently bound per 1 g purified alginate in a 1% alginate solution. Peptide incorporation efficiency can be quantified using the Pierce Micro BCA assay. One of ordinary skill in the art would readily use other coupling methods, e.g. the carboxyl groups of each mannuronic acid monomer can be modified by attachment of amino groups found on proteins using covalent alginate-protein/peptide coupling chemistry.

### Dry Scaffold Generation

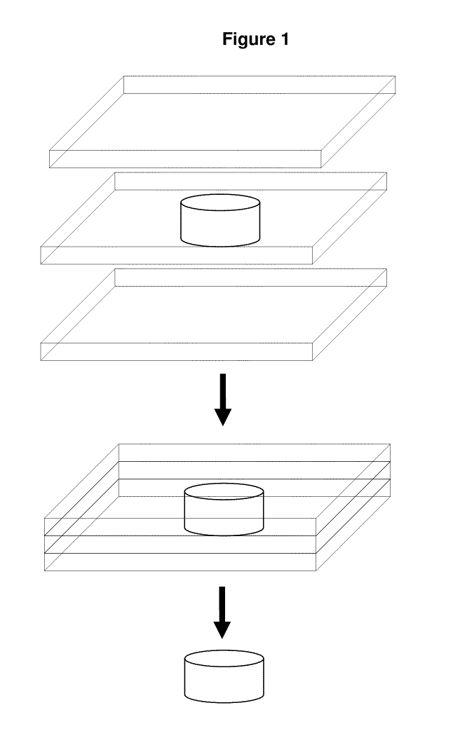

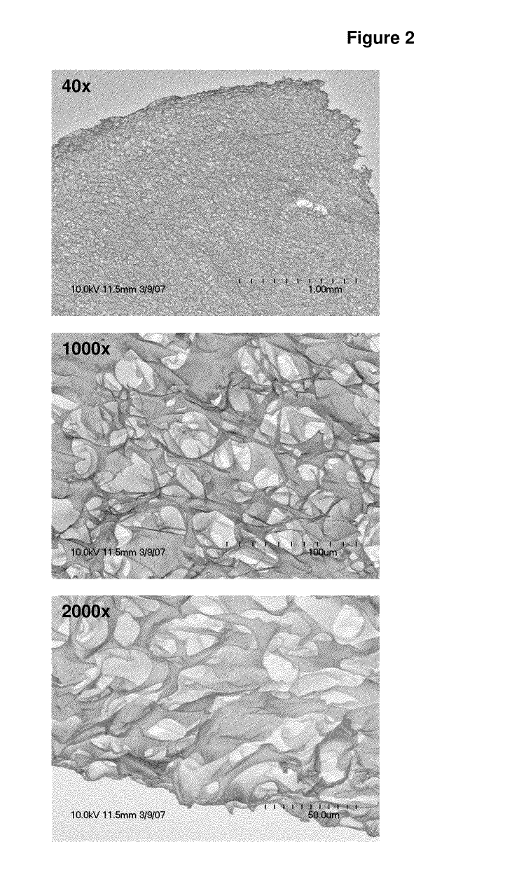

In one embodiment, RGD-alginate solution was cast between two 40 durometer 0.030″ thick silicone sheets (Specialty Manufacturing), frozen at −20° C. and transferred to 1.1% calcium chloride solution in 70% ethanol in ddH₂0 at −20° C. to solidify. This process creates a highly porous 3-dimensional scaffold. This method was superior to lyophilization because it prevents the formation of an impenetrable surface skin on the scaffold surface. Resulting scaffolds were washed in ddH₂O, followed by 100% ethanol and dried in air or by using filter paper in low adhesion tissue culture plastic plates (FIGS. 1-2).

Dry scaffolds can be loaded with cells by submerging in a cell suspension or cells were directly applied onto the scaffold. Cells were absorbed due to the hygroscopic nature of the cyclic RGD-alginate matrix. After absorption of cells, cell-scaffolds were kept in culture medium for in vitro studies or implanted in specific sites in vivo.

Dry scaffolds can be loaded with bioactive molecules such as proteins or pharmacological compounds for sustained release, for example growth factors to promote scaffold vascularization or immunomodulatory compounds to promote cell survival or after implantation. Implantation sites include subcutaneous, intramuscular, intraperitoneal, intrathoracic, subscapular, and intraomental as well as intraorgan under some conditions. Dry scaffolds are typically used for chronic cardiac ischemia, but can be used for different purposes.

### Wet Scaffold Generation

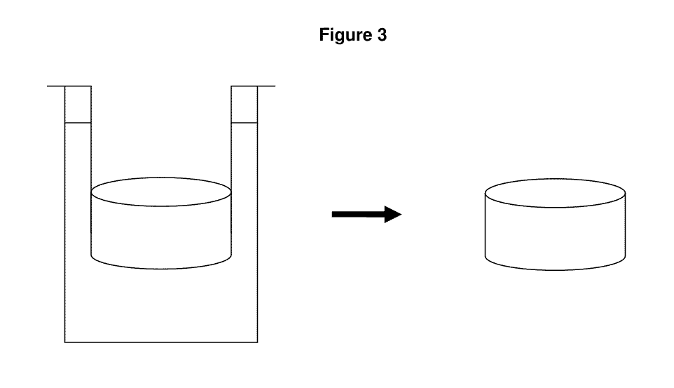

In one embodiment, RGD-alginate solution mixed with or without cells and/or bioactive compounds can be cast in top wells of tissue culture trans-wells with 1.1% calcium chloride in the bottom well and incubated for 20 minutes using cell-containing solution or overnight without cells. Incubation results in solidification of RGD-alginate (FIG. 3). Circular scaffolds without cells are washed in ddH₂O and kept wet and sterile until implantation. Cell-containing scaffolds were washes in buffers without calcium binding or calcium chelating salts.

Wet scaffolds can be loaded with cells and/or growth factors before solidification or cells were injected into the scaffold before or after implantation. Growth factors in the scaffold provide signals to establish a vascular network throughout the scaffold before cells are injected in vivo, which improves survival of injected cells. Macroporous channels of various sizes (100-300 micrometers) can be generated using wiring in order to increase the permeability of the scaffold. Wet scaffolds are typically used for pancreatic islet transplantation in a diabetic model, but can be used for different purposes such as enzyme deficiency diseases, liver failure or immunological manipulation of the host.

### Immunological Modification

In one embodiment, liquid alginate can be mixed with immunomodulatory compounds, e.g. synthetic drugs, peptides, antibodies, immunomodulatory cells and enzymes, cytokine secreting cells, antibody secreting cells or Sertoli cells. After scaffold formation and implantation, compounds are released in a sustained manner to prevent rejection of cells or tissue present in the scaffold. Alternatively, immunomodulatory compounds are covalently bound to liquid alginate without sustained release to act locally in the scaffold after implantation. Initial substances to introduce will include, but are not limited to, ILT-3, Fas ligand, CTLA4 IgG, anti-CD40, anti-CD45, anticomplement compounds and/or L-Dopa.

### Mode of Scaffold Implantation

Scaffold containing different compounds may be seeded with the cells in vitro and then implanted into tissue. Another option is implanting the scaffold days or weeks before cells transplantation which permits appropriate preconditioning of the transplant “bed” including its prevascularization and immunomodulation, leading to improved cell engraftment and survival. Implantation sites include subcutaneous, intramuscular, intraperitoneal, intrathoracic, subscapular, and intraomental as well as intraorgan under some conditions.

### Uses of The Invention

Scaffolds can be loaded with different cell types, for example stem cells or pancreatic islets, and/or bioactive compounds and implanted at sites to promote vascularization, tissue and cell regeneration and modulate the local immune response. The cyclic RGD peptide promotes vascular formation of the host tissue, cell binding and survival of seeded cells. In vitro, cyclic RGD peptide promotes cell survival more efficiently than linear RGD peptide, possibly due to increased stability, resistance to protease degradation and stronger affinity for the receptors, which results in improved live cell numbers after prolonged culture.

Scaffolds with growth factors but without cells can be implanted in order to create optimal local conditions, i.e. a prevascularized and immunomodulated “bed” into which cells are transplanted at a later time point, for example pancreatic islets, hepatocytes, ovarian cells and other appropriate cells in the submuscular, intramuscular, intraomental or subcutaneous space. Modified alginate may be injected in combination with cells and/or growth factors directly into tissue in order to provide cell survival and retention. In the case of cell transplantation without carriers (i.e. scaffolds) for degenerative diseases, cell transplantation is hampered by very low survival of transplanted cells, due to the absence of adhesion molecules and sufficient blood supply in the host tissue, especially when ischemia is present. Implantation of cells and/or bioactive compounds in combination with this scaffold might overcome this problem. Due to the purity of the material, which prevents an immune response or sensitization of the host, and the fact that the material is non-animal derived, which eliminates the risks of pathogen transfer, clinical application of the scaffold as a carrier material for active compounds and transplanted cells is potentially possible.

The present invention provides a porous three dimensional scaffold comprising purified alginate molecules that are conjugated to cyclic RGD peptides. In one embodiment, the purified alginate molecules are poly-mannuronic acid molecules or poly-guluronic acid molecules. In general, the poly-mannuronic acid molecules can be derived from seaweed, e.g. the giant kelp Macrocystis pyrifera, Ascophyllum nodosum and various types of Laminaria etc. In one embodiment, the cyclic RGD peptides comprise a sequence RGDxy, wherein “x” is D-phenylalanine or D-tyrosine, and “y” is L-cysteine, L-glutamic acid, L-lysine or L-valine. In one embodiment, the alginate molecules are purified to contain less than 0.305% protein. In another embodiment, the alginate molecules are purified to contain less than 12.5 EU endotoxin per gram dry alginate, or less than 1.0 μg DNA per gram dry alginate, or less than 10.0 μg RNA per gram dry alginate.

In one embodiment, the porous three dimensional scaffold of the present invention further comprises cells such as stem cells, myocytes, human bone marrow derived mesenchymal precursor cells, or islet cells. In another embodiment, the scaffold of the present invention comprises one or more immunomodulatory factors or growth factors. Examples of such factors include, but are not limited to, antibodies, immunomodulatory peptide, synthetic drug, growth factors such as PDGF, VEGF or thymosin beta 4 etc. In yet another embodiment, the scaffold of the present invention comprises cells and one or more of the above described factors.

The present invention also provides a composition comprising the porous three dimensional scaffold of the present invention.

The present invention also provides a porous three dimensional scaffold comprising purified alginate molecules, wherein the alginate molecules are purified by a method comprising the steps of: dissolving the alginate molecules in an acidic; and removing protein, DNA, RNA and endotoxin contamination by neutral and active charcoal treatment, filtration through bioactive filter membranes and precipitation with ethanol. In one embodiment, the alginate molecules are purified to contain less than 0.305% protein. In another embodiment, the alginate molecules are purified to contain less than 12.5 EU endotoxin per gram dry alginate, or less than 1.0 μg DNA per gram dry alginate, or less than 10.0 μg RNA per gram dry alginate.

The present invention also provides a method of promoting tissue or cell transplantation, comprising the steps of: preparing a porous three dimensional scaffold disclosed herein; loading the porous three dimensional scaffold with cells or tissue; and transplanting the loaded porous three dimensional scaffold into a human or animal, thereby obtaining better transplantation results as compared to transplantation without the porous three dimensional scaffold. In one embodiment, the three dimensional scaffold further comprises one or more of the above described immunomodulatory factors or growth factors.

The present invention also provides a method of promoting tissue or cell transplantation, comprising the steps of: creating a vascular bed by transplanting a porous three dimensional scaffold disclosed herein into a human or animal; and transplanting cells or tissues into the vascular bed, thereby obtaining better transplantation results as compared to transplantation without using the porous three dimensional scaffold. In one embodiment, the three dimensional scaffold further comprises one or more of the above described immunomodulatory factors or growth factors.

The present invention also provides a method of promoting cell transplantation to heart, comprising the steps of: preparing a porous three dimensional scaffold disclosed herein; loading the porous three dimensional scaffold with stem cells or myocytes; and transplanting the loaded porous three dimensional scaffold into a heart, thereby obtaining better transplantation results as compared to transplantation without the porous three dimensional scaffold. In one embodiment, the porous three dimensional scaffold further comprises one or more immunomodulatory factors or growth factors (such as PDGF, VEGF, or thymosin beta 4).

The present invention also provides the porous three dimensional scaffold disclosed herein for uses as a medicament for promoting tissue or cell transplantation. In one embodiment, the porous three dimensional scaffold loaded with cells or tissue was transplanted into a human or animal, thereby obtaining a better transplantation result as compared to transplantation without the porous three dimensional scaffold. In another embodiment, the three dimensional scaffold further comprises one or more of the above described immunomodulatory factors or growth factors.

The present invention also provides the porous three dimensional scaffold disclosed herein for uses as a medicament for promoting tissue or cell transplantation. In one embodiment, a vascular bed is created by transplanting a porous three dimensional scaffold disclosed herein into a human or animal, and cells or tissues are then transplanted into the vascular bed, thereby obtaining a better transplantation result as compared to transplantation without using the porous three dimensional scaffold. In another embodiment, the three dimensional scaffold further comprises one or more of the above described immunomodulatory factors or growth factors.

The invention will be better understood by reference to the Examples which follow, but those skilled in the art will readily appreciate that the specific experiments detailed are only illustrative, and are not meant to limit the invention as described herein, which is defined by the claims which follow thereafter.

Throughout this application, various references or publications are cited. Disclosures of these references or publications in their entireties are hereby incorporated by reference into this application in order to more fully describe the state of the art to which this invention pertains.

### Example 1

### RGD-Modified Poly-Mannuronic Acid Substrate For Cell Transplantation

Alginate is the descriptive name for polysaccharides that can be derived from several species of seaweed, including the giant kelp Macrocystis pyrifera, Ascophyllum nodosum and various types of Laminaria. It is composed of poly-mannuronic or poly-guluronic acid. Poly-mannuronic acid chains have a linear structure, while poly-guluronic acid chains are buckled. In one embodiment of the present invention, alginate will refer to alginate purified according to the method disclosed above.

Alginate is soluble in water and solidifies in the presence of calcium ions. It is biodegradable, non-toxic and in solid form does not provide mammalian cell adhesion motifs. It can be injected as a liquid or implanted as a 3D scaffold. The carboxyl groups of each mannuronic acid monomer can be modified by attachment of amino groups found on proteins using covalent alginate-protein/peptide coupling chemistry.

Raw alginate is heavily contaminated and needs to be purified before it can be implanted into living organisms to prevent rejection reactions from the host. A custom purification protocol described above was developed to render alginate free from mitogenic activity. Protein levels were decreased to less than 3.05 mg protein per gram alginate (0.305%), DNA to less than 1 μg per gram alginate and RNA to less than 10 μg per gram alginate.

### Integrin Binding Peptides

Integrin binding peptides are small chains of amino acids that contain the Ag-Gly-Asp (RGD) sequence, which binds to integrin receptors αVβ3 and α5β1 on the cell surface. These peptides block cell adhesion in solution because they block interaction of integrin receptors with a solid substrate. When RGD peptides are immobilized on a solid substrate, they promote adhesion by binding to integrin receptors. Many cell types use RGD-integrin interaction to adhere to a solid substrate. After binding, integrin receptors get activated and promote cell survival by intracellular signaling via AKT.

Integrin binding peptides are synthetically fabricated and can have several different sequences, which changes their biochemical properties. Cyclic RGD peptides (for example, cRGDfK or GPenGRGDSPCA) have been designed that are more stable in solution than linear peptides and bind to integrin receptors with higher affinity. The GPenGRGDSPCA peptide has a disulfide bridge between Pen-2 and C-9 (cysteine at position 9), which results in cyclicalization of the peptide, rendering it 30× more stable in aqueous solutions. Pen refers to penicillamine, which is incorporated in peptides to stabilize the structure by interacting with cysteine through disulfide bonding. The number of peptides or integrin binding peptides that bound to alginate can be regulated by the use of different concentrations of coupling reagents.

### Cell Embedding

In one embodiment, cells can both be embedded in alginate before or after solidification. Cells can be resuspended in alginate, after which the alginate is solidified by calcium ions using a transwell system. This creates a 3D alginate/cell structure in which cells are immobilized without space to migrate.

In another embodiment, alginate can also be formed into a 3D porous scaffold. Freeze gelation results in 3D scaffolds with open pore structure, including on the surface of the scaffold. Cells can be added to this matrix after solidification and drying, and have space to migrate since scaffold pores are 50-200 μm.

For porous scaffold fabrication, alginate was cast in silicone molds (16 mm×0.75 mm, but can be any size) and solidified at −20° C. After 24 hours of solification, scaffolds were removed from molds and calcium chloride 1.1% in 70% ethanol was added at −20° C. Scaffolds were incubated for another 24 hours at −20° C. This resulted in porous scaffolds with open pore structure, since silicone prevents surface skin formation due to its negative charge.

### Cell Survival

Solid RGD modified alginate can be used to grow adherent cells on its 2D surface. After seeding cells on RGD modified alginate, cells will spread and remain viable due to the RGD sequence, whereas cells seeded on unmodified alginate will not adhere, clump together and die.

Cells seeded inside 3D alginate scaffolds show significant dose dependent improvement of survival after modification of alginate with RGD peptides, both after embedding and after seeding in 3D scaffolds.

Embedding results in close contact between the RGD modified alginate and cell surfaces, resulting in integrin signaling and improved survival. In one embodiment, survival and adhesion was assessed using stro-3 positive human bone marrow derived precursor cells. Cell survival increased from 8% (0 mg/g GPenGRGDSPCA peptide) to 52% (10 mg/g GPenGRGDSPCA peptide).

In another embodiment, survival and adhesion was assessed using rat neonatal fibroblasts, rat neonatal cardiomyocytes and stro-3 positive human bone marrow derived precursor cells. At 1 week, neonatal rat myocyte viability inside scaffolds increased from 3.3±1.2% (0 mg/g cRGDfK) to 12.3±0.1% (10 mg/g cRGDfk) to 28.9±7.3% (10 mg/g cRGDfk+gelatin) (P<0.05). Clusters of beating myocytes could be detected in the scaffolds. Neonatal rat cardiac fibroblast viability increased from 48.8±21% (0 mg/g cRGDfK) to 77.2±3.2% (10 mg/g cRGDfk) (P<0.05). Human bone marrow mesenchymal precursor cell survival increased from 8.3±0.4% (0 mg/g cRGDfK) to 33.3%˜5.3% (2 mg/g cRGDfk) to 61.0%˜4.2% (20 mg/g cRGDfK) (P<0.05). Cyclic RGD peptide modified alginate has consistently shown higher survival rates than linear RGD peptides, both of embedded cells and cells seeded in porous 3D scaffolds.

### Immunogenicity of Custom Purified Alginate In Vitro

Low molecular weight alginate composed mainly of poly-mannuronic acid (Sigma 0682) was purified using a custom protocol described herein. Immunogenicity was compared to commercially available Ultrapure Alginate (LVM, LVG, FMC/Novamatrix) preparations, and unpurified alginate (Sigma 0682). Protein contamination of custom purified alginate was ˜3.05 mg/g alginate, whereas unpurified levels were 10.5 mg/g. Ultrapure commercial preparations (LVM and LVG) contained ˜4.5 mg/g protein per gram alginate, as determined by micro BCA assay. Endotoxin was determined by LAL assay (Pyrosate, detection limit 0.25 EU/ml) and was negative, indicating endotoxin contamination <12.5 EU endotoxin/g alginate. In vitro immunogenicity was determined using the rat splenocyte proliferation assay. Splenocyte proliferation of custom purified alginate after 1 week in culture was comparable to negative control (growth medium without alginate). Unpurified alginate from the same batch and Ultrapure alginate preparations induced a significant increase in splenocyte proliferation, suggesting mitogenic contamination.

### Scaffold Implantation

Porous 3D alginate scaffolds were applied to ischemic myocardium of nude rats 4 weeks following ligation of the left descending coronary artery. Scaffold remained attached to the epicardial surface for 2 weeks and induced vascular formation.

### Scaffold Perfusion

Solid 3D alginate scaffolds were implanted between the abdominal muscles of rats for 30 and 60 days. Scaffold perfusion was measured using microbubbles in combination with Doppler ultrasound detection. Scaffold perfusion could be determined in vivo and was comparable to surrounding tissues. Immunohistochemistry confirmed these results by abundant capillary and arteriole formation inside the scaffold.

### Example 2

### Three-Dimensional RGD Peptide Modified Alginate Scaffold Seeded with Cells for Cardiac Repair Following Myocardial Infarction

Stem cells can be directly injected into damaged heart tissue to generate new vessels and salvage myocardium (Martens et al., 2006). For example, intra-myocardial injection of human bone marrow derived mesenchymal precursor cells (hMPCs) positive for the mesenchymal stem cell marker Stro-1 has previously been shown to induce angiogenesis in ischemic rat myocardium, resulting in global improvement of myocardial function. However, in humans, placebo controlled trials using autologous whole bone marrow cell therapy for acute myocardial infarction have yielded mixed results with either little or no beneficial effects (Schachinger et al., 2006; Lunde et al., 2006). The cause of this discrepancy is unclear and might lie in the lack of retention (Teng et al., 2006) or survival of transplanted cells. Indeed, in animal studies, only 0.1% live cells could be detected in the rat heart by PCR 48 hours post myocardial infarction.

One method to increase survival of transplanted cells in the myocardium is by creating a local microenvironment that promotes angiogenesis and retention of cells, for example by delivering myoblasts using injectable fibrin scaffolds (Christman et al., 2004) or implanting rat myocytes in engineered collagen (Zimmermann et al., 2006; Kutschka et al., 2006) or alginate (Leor et al., 2000) grafts. Using a different approach, transplantation of a mono-layered interconnected mesenchymal stem cell patch on the infarct scar has been shown to regenerate myocardium after myocardial infarction in rats (Miyahara et al., 2006).

Based on the aforementioned studies, a new design for a versatile and clinically applicable material for cell transplantation (e.g. hMPCs) that would increase cell survival after transplantation is described below.

Ligand activation of integrin αVβ33, which is expressed on most cells (e.g. hMPCs), is known to promote angiogenesis and to protect against apoptosis. In solution, synthetic peptides containing the amino acid sequence Arg-Gly-Asp (RGD) competitively bind and activate αVβ3 on the cell surface but block its function. However, once immobilized on a solid surface or in a 3-dimensional scaffold, RGD peptides provide a substrate for cells that promotes cell viability (Nuttelman et al., 2005). Mannuronic-acid rich alginate is a non-toxic, biocompatible hydrogel without mitogenic activity that can be solidified under physiological conditions by adding divalent ions like Ca²⁺ (Klock et al., 1997). In its unmodified state, human cells can not adhere to alginate, because it consists of negatively charged polysaccharide chains. However, alginate can be chemically modified with adhesion molecules such as RGD peptides to create a suitable microenvironment for cells such as mesenchymal stem cells (Markusen et al., 2006). The addition of growth factors to RGD modified alginate hydrogel has further been shown to have additional beneficial effects on myoblast survival and proliferation (Hill et al., 2006a; Hill et al., 2006b). PDGF-bb and VEGF stabilize induced vascular networks in Matrigel assay (Chen et al., 2007) and 3 dimensional scaffold based culture in vivo (Chen et al., 2007; Kano et al., 2005).

Hence, it is hypothesized that activation of αVβ3 by an immobilized RGD peptide in a 3-dimensional mannuronic-acid rich alginate scaffold in combination with PDGF-bb and VEGF will improve cell viability in the scaffold in vitro and in vivo. Since many human cell types express αVβ3, RGD modified mannuronic-acid rich alginate may be used in combination with different cell types to improve their survival after transplantation.

In one embodiment, stem cells with or without growth factor containing grafts were implanted to examine their effects on cell survival and cardiac function after myocardial infarction in vivo. The effects can be compared to empty grafts, grafts with PDGF-bb, b-FGF and VEGF alone and to stem cells directly injected into the myocardium.

### Experiment 1

Scaffold preparation. For alginate purification, low molecular weight alginate (Sigma 0682) composed primarily of 1,4-poly-mannuronic acid at a concentration of 1.5% in 10 mM phosphate buffer, pH 5.5 at 20 degrees celcius in ddH2O was dissolved and treated with neutral carbon 1.5% for 24 h at 50 degrees celcius, filtered through glass pre-filters and treated with active carbon 1.5% for 24 h at 50 degrees celcius and filtered through glass pre-filters. After glass pre-filter filtration, the solution was kept at 4 degrees Celcius for 24 hours. Subsequently, the solution was filtered through hydrophobic Immobilon P membranes (50 ml per membrane in 90 mm Buchner funnel). After purification, Pierce micro BCA was used to determine the presence of protein. Qubit (Invitrogen) was used for DNA or RNA determination. Endotoxin presence was determined by using the Pyrosate kit (Cape Cod).

Using carbodiimide chemistry (EDC and sulfo-NHS in MES buffer, Pierce), linear or cyclic RGD peptides (American Peptide Co.) was covalently bound according to Rowley et al. (1999). Peptide incorporation efficiency was quantified using the micro BCA assay. Sixty μl RGD-alginate solution was casted between silicone sheet molds (16 mm×0.75 mm), frozen at −20° C. and transferred to 70% ethanol in ddH20 at −20° C. to solidify, creating a highly porous disc. Discs were washed in ddH2O, air dried, placed in 12-well plates and loaded with growth factors. Retention and time course release of growth factors can be measured by Pierce micro BCA. Dry discs can be seeded with 2×10⁶ cells in 15 μl full medium consisting of αMEM supplemented with 10% FCS, 0.1% BSA, ascorbic acid 10-4 M, mercaptoethanol 10-4 M and 0.2% primocin (Amaxa). After seeding one side of the disc, it was inverted and after 5 minutes, 2×10⁶ cells in 15 μl were applied. Due to the interconnected macroporous (100-200 μm pore size) and hygroscopic nature of the discs, cells were absorbed and distributed evenly throughout the scaffold. After 15 minutes of incubation at 37° C. in humidified room air and 5% CO2, 1 ml of either full medium or full medium without 10% FCS (serum free medium) was carefully added and the discs were kept at 37° C. in humidified room air and 5% CO2. For in vivo studies, the discs were prepared as described, loaded with cells in full medium and washed gently with PBS before implantation.

Cell viability studies. Cells seeded scaffolds were incubated in full medium and serum free medium at 37° C. in room air and 5% CO₂ and at 37° C. in anaerobic conditions (BD Gaspak System) for different time points. Viability can be determined by trypan blue exclusion assay and flow cytometry using propidium idodide and the Live/Death assay (Invitrogen). Apoptosis can be determined using TUNEL technique. Pre-treatment of cells with anti-αVβ3 mAb or soluble RGD peptides in both unmodified and RGD-modified scaffolds can be used as controls. After culture, cells were recovered from scaffolds using citric acid/EDTA buffer and subsequently, viability will be determined as described before.

Alternatively, passage 2 or 4 hMPCs can be purified using Stro-1 mAb and magnetic microbeads (Miltenyi). Stro-1 expression can be evaluated by flow cytometry surface staining using anti-Stro-1 mAb; cell populations >90% positive for Stro-1 were used. hMPC seeded scaffolds were incubated in full medium and serum free medium at 37° C. in room air and 5% CO2 and at 37° C. in anaerobic conditions (BD Gaspak System) for different time points. Viability can be determined by trypan blue exclusion assay and flow cytometry using propidium idodide and the Live/Death assay (Invitrogen). Apoptosis can be determined using TUNEL technique. Pre-treatment of cells with anti-αVβ3 mAb or soluble RGD peptides in both unmodified and RGD-modified scaffolds can be used as controls. After culture, hMPCs were recovered from scaffolds using citric acid/EDTA buffer and subsequently, viability was determined as described before.

³H-thymidine incorporation. Solid disc cultures can be incubated in the presence of 3H thymidine at a final concentration of 5 μCi/m1 for different time points, washed (3×15 min) with αMEM full medium, and frozen until analysis. ³H-thymidine incorporation can be measured using a liquid scintillation counter. Aliquots can also be prepared for PicoGreen DNA quantitation assays.

Animals, surgical procedures and implantation of human cells. Rowett (rnu/rnu) athymic nude rats (Sprague-Dawley, Indianapolis, Ind.) were used in studies approved by the Columbia University Institute for Animal Care and Use Committee. After anesthesia, a left thoracotomy was performed, the pericardium opened and the left anterior descending (LAD) coronary artery was either ligated or left intact (sham procedure). After 4 weeks, cells in 50 μl PBS were injected intra-myocardially at 5 sites in the infarct border zone or cell seeded scaffolds with 2×10⁶ cells+PDGF and VEGF, scaffolds containing PDGF, bFGF and VEGF alone or empty scaffolds can be placed on the epicardium covering the infarct scar and infarct border zones. Animals were sacrificed at 4, 8, 12 and 24 weeks after transplantation. Cardiac function can be assessed by hemodynamics (Millar) and echocardiography. Cell/scaffold integration, myocyte regeneration/cycling and neo-angiogenesis can be assessed by immunohistochemistry.

Analyses of myocardial infarction. Echocardiographic studies can be performed in all rats at baseline, 4 weeks after myocardial infarction, and at 4, 8 and 12 weeks after implantation of cell seeded scaffolds, cell populations or saline. Echocardiography can be performed using a high frequency linear array transducer (Visual Sonics Vevo 770 Micro Imaging System). 2D images were obtained at mid-papillary and apical levels. End-diastolic (EDV) and end-systolic (ESV) left ventricular volumes were obtained by bi-plane area-length method, and % left ventricular ejection fraction was calculated as [(EDV-ESV)/EDV]×100. Cardiac output (CO) was measured using an ultrasonic flowprobe and cardiac index calculated as CO per weight.

Myocardial and scaffold perfusion can be quantified using untargeted micro bubbles (Visual Sonics). All echocardiographic studies were performed by a blinded investigator. For hemodynamic measurements, animals were cannulated via the right carotid artery and pressure volume loops were obtained using a Millar micro catheter and analyzed using Chart for Windows.

Cell survival after transplantation. 4, 8, and 12 weeks after transplantation, whole hearts can be harvested. Hearts were homogenized, DNA was extracted using a DNA extraction kit (Roche), human DNA was quantified using qPCR using human beta globin primers and compared to a standard curve to estimate the total number of live human cells in the heart.

Scaffold perfusion. Isolated hearts can be perfused with Evans blue at the aortic root to determine communication between host vasculature and scaffold neo-vasculature. After perfusion with PBS, hearts were perfused at 100 mm Hg with 4 mg/ml Evans blue in PBS for 30 seconds. Within 1 minute after perfusion, photographs were taken using a digital camera (Nikon D50). Hearts were then be washed and used for histological analysis.

Histology and immunohistochemistry. For in vitro studies, alginate cultures were fixed overnight in 10% buffered formalin to which 100 mM CaCl₂ was added to prevent alginate depolymerization. Once fixed, the alginate discs were removed from the culture well and paraffin embedded. Serial cross sections 3 μm in thickness were cut from the center of each of the alginate discs. Scaffold cellularity can be determined using routine haematoxylin/eosin (H&E) staining expressed as cell number per high power field (400×). Proliferation can be determined with anti-Ki67 mAb and anti-PCNA mAbs. Apoptotic cells can be identified with anti-caspase-3 mAb. Detection can be done using an HRP-conjugated anti-mouse IgG secondary antibody with diaminobenzidine as substrate, according to the manufacturer's instructions. Sections were counterstained with haematoxylin.

In histological studies, following excision, whole hearts from each experimental animal were sliced at 10-15 transverse sections from apex to base. Representative sections were fixed in formalin and stained for routine histology (H&E) to determine scaffold integration in the host tissue and cellularity of the scaffold expressed as cell number per high power field (400×). Cell survival was determined by measuring the area covered by cells that stain positive for human MHC class I using ImageJ software (NIH). Cell area can be reported as percentage of scaffold area. A Masson's trichrome stain can be performed, which labels collagen blue and myocardium red, to evaluate collagen content on a semi-quantitative scale (0-3⁺), with 1+ light blue, 2+ light blue and patches of dark blue, and 3+dark blue staining. This enables measurement of the size of the myocardial scar and potential fibrosis of the scaffold using a digital image analyzer. The lengths of the infarcted surfaces, involving both epicardial, endocardial and scaffold regions, can be measured with a planimeter digital image analyzer and expressed as a percentage of the total ventricular circumference. Final infarct and scaffold sizes can be calculated as the average of all slices from each heart. All studies were performed by a blinded pathologist. Infarct and scaffold sizes were expressed as percent of total left ventricular area. Final infarct and scaffold sizes can be calculated as the average of all slices from each heart.

Integration of cell/scaffold in host myocardium. Since it has previously been shown that stem cells can induce vascular network formation and might be able to differentiate into vascular structures, capillary density and species origin of the capillaries and arterioles can be quantified in the myocardium and in the scaffold by staining with mAbs directed against von Willebrand's factor, rat or human CD31, rat or human MHC class I and rat or human α-smooth muscle actin. Staining can be performed by immunoperoxidase technique using an avidin/biotin blocking kit, a rat-absorbed biotinylated anti-mouse IgG, and a peroxidase-conjugate. Capillary density can be determined from sections labeled with anti-von Willebrand's factor mAb at 4, 8, 12 and 24 weeks post infarction and compared to the capillary density of unimpaired myocardium and scaffold. Values are expressed as anti-von Willebrand's factor positive cells per HPF (400×).

Cardiomyocyte regeneration can be measured by immunohistochemistry of tissue sections, as outlined above for glass slides, determining the proportion of cells co-staining for α-sarcomeric actinin and Ki67 or BrdUrd after feeding the animals BrdUrd ad libitum. Cardiomyocyte apoptosis can be measured by immunohistochemistry of tissue sections, as outlined above for glass slides, using TUNEL technique and staining for cardiomyocyte markers to determine the proportion of cardiomyocytes with apoptotic nuclei.

### Histology of Epicardial Scaffold Application

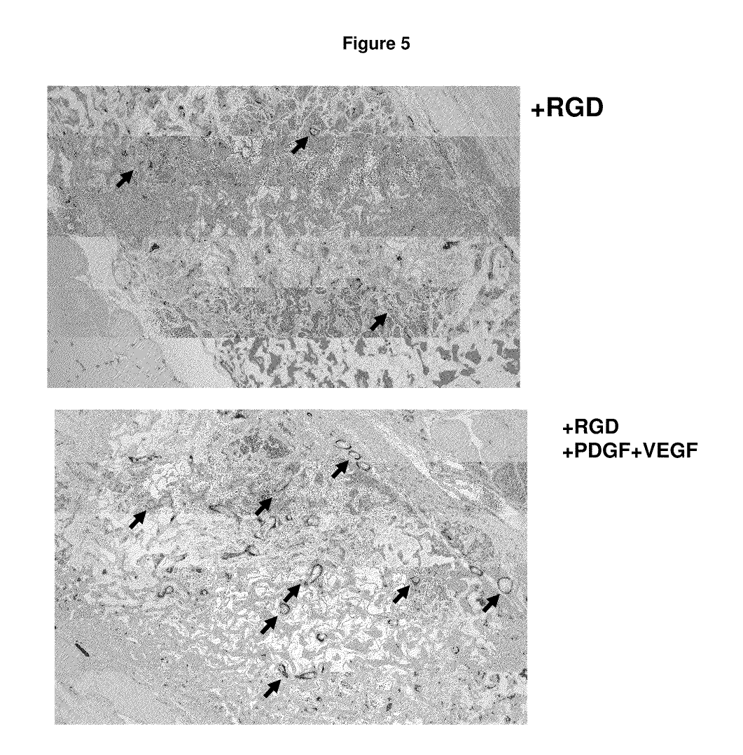

cRGDfK scaffolds (20 mg cRGDfK per gram alginate) seeded with human mesenchymal precursor cells were applied to the epicardium 2 days after myocardial infarction and harvested for histology after 1 week. Staining was done for fibrosis (Masson's trichrome) and endothelial cells (fVIII). Scaffolds can be identified on the epicardium (labeled S). Vascular formation was most evident in the border zones of the infarcted heart (FIG. 5).

### Cardiac Function Following Scaffold Application

Myocardial infarction was induced in nude rats via thoracotomy and permanent ligation of the left anterior descending artery. Two days later, rats were re-operated and hearts were injected with saline or cRGDfK (20 mg per gram alginate) modified scaffold seeded with 1×10⁶ human mesenchymal precursor cells. One week later echocardiograms were performed and fractional shortening was determined. Scaffold implantation resulted in preservation of cardiac function compared to saline injections. After 1 week, fractional shortening in the saline injected group decreased by 15.2%±2.5%, whereas the decrease was 1.23%±12.2% in the RGD scaffold treated group.

### Experiment 2

Three dimensional scaffolds were generated by freezing cyclic RGDfK alginate solution between silicone sheets to generate highly porous scaffolds (16×0.75 mm), followed by immersion in 70% ethanol/1.1% CaCl₂ solution at −20° C. to solidify and dried at room temperature (freeze gelation method).

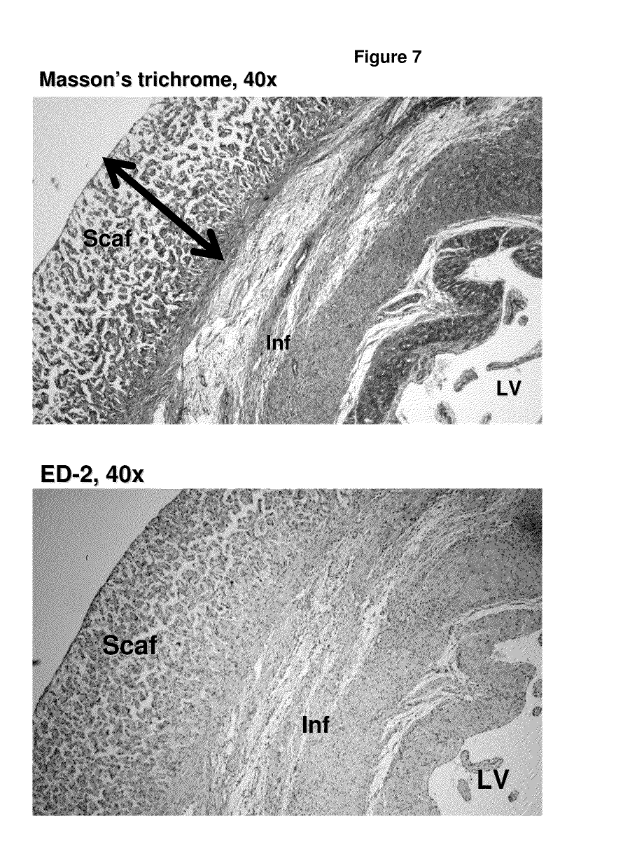

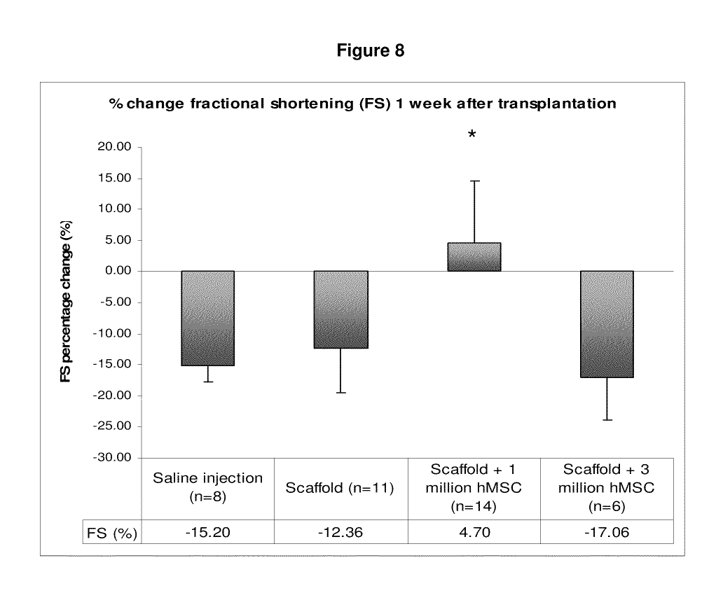

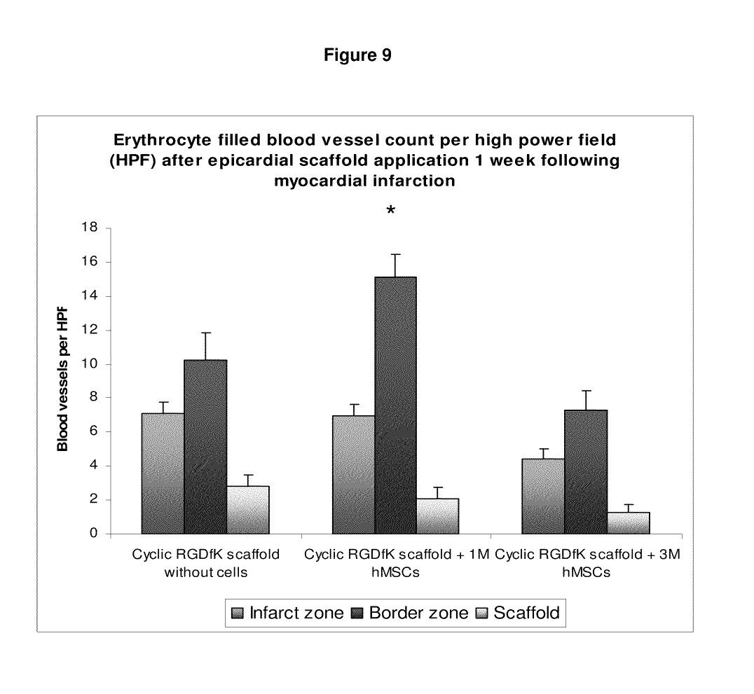

One million neonatal rat cardiomyocytes, neonatal rat cardiac fibroblasts or human bone marrow mesenchymal stem cells (hMSCs) were seeded into three dimensional scaffolds. Cell viability in vitro was determined by WST-1 and trypan blue exclusion assays 1 week later. In vivo, scaffolds were used 24 hours after seeding. Scaffolds with 1 million or 3 million hMSCs were applied to the epicardium of athymic (rnu/rnu) nude rats 48 hours following left coronary artery ligation. Scaffold biocompatibility was determined by ED-2 staining for macrophages. Angiogenesis was determined by blood vessel formation in the infarct zone and the border zone of the MI, and in scaffolds. Cardiac function was determined by fractional shortening (FS) using echocardiography. All in vivo analyses were performed 1 week after scaffold transplantation. The data were shown in FIGS. 7-9. At 1 week post-transplantation, hMSC-seeded cyclic RGDfK peptide-modified scaffolds demonstrated cellularization and vascular in-growth, indicating engraftment to host myocardium. No immune response was observed. Cyclic RGDfK modified scaffolds seeded with 1 million hMSCs increased vessel formation in the infarct border zone and improved cardiac function following epicardial application, whereas unseeded scaffolds and scaffold seeded with 3 million cells had no effect.

### Discussion

Cell therapy for cardiovascular disease may become a viable alternative to currently established therapies. However, an important obstacle for successful cell-based therapies is the low engraftment and viability of transplanted cells. The development of tissue engineered matrices for the delivery and support of transplanted cells might overcome this problem. Mannuronic-rich alginate was picked to meet the requirements for clinical application and, in its unmodified form, is FDA approved and used in the clinic as a wound dressing material for decades. The alginate matrix serves as a blank 3-dimensional canvas and can be easily modified with biologically active peptides. It is therefore extremely versatile for mechanistic studies focusing on the effect of particular peptides on cells in 3-dimensional tissue grafts. From a translational point of view, it is important to know the nature of the transplanted material in order to understand its effects on grafted cells and the host.

The effects of RGD peptide modified alginate based cell delivery on cell survival and cardiac function after myocardial infarction has not been previously investigated. In preliminary studies, it have been found that certain RGD peptides are more effective in promoting cell viability than others. It is expected that modification of alginate with the optimal RGD peptide will enhance cell survival after myocardial implantation and that the effects on cardiac regeneration and angiogenesis will be superior compared to intramyocardial cell injections. Cell seeded scaffolds are expected to induce cardiac angiogenesis by either direct contribution of mesenchymal precursor cells to the vasculature as pericytes or by paracrine effects by vascular growth factor production. These effects are expected to salvage ischemic myocardium, leading to increased cardiac myocyte survival, decreased apoptosis and overall improvement in cardiac function.

Biocompatible scaffolds are an attractive approach for cell transplantation to repair damaged tissue. Cell dose can be controlled before transplantation and cell loss can be kept to a minimum since the “stickiness” of biomaterial promotes local retention of cells compared to direct cell injection.

Cyclic RGDfK peptide-modified alginate enhanced cell viability over time compared to linear GRGDSP peptide-modified alginate. This is likely due to higher stability of cyclic RGDfK peptide, which prevents spontaneous and proteolytic degradation, making it more readily available to bind to integrin receptors on the cell surface. Cyclic RGDfK peptides bind to integrin receptors with higher affinity than linear RGD peptides, which may further enhance survival signaling.

Cell death of seeded cells after transplantation may contribute to deleterious effects on tissue regeneration. Since the scaffold is not vascularized at the time of transplantation, oxygen and nutrients are initially delivered by diffusion. RGD-modification may enhance vessel growth by promoting endothelial cell proliferation, but an existing vascular network would be more desirable, may decrease cell death and enhance regenerative effects.

Scaffolds seeded with 1 million hMSCs increased blood vessel formation in the border zone of the infarct and augmented cardiac function. This effect was not observed using scaffolds with 3 million hMSCs. An explanation for this result may lie in the paracrine factors (i.e. cytokines) that hMSCs secrete, such as IL-6 and MCP-1. Local excess cytokine concentrations may be toxic to cardiac myocytes and abolish the beneficial effects.

Alternatively, a high number of cells inside scaffolds may lead to accelerated cell death in vivo due to an initial lack of oxygen and nutrients, abrogating the beneficial effects. Because cell dose can be controlled to a greater extend and cell loss decreased to a minimum compared to direct cell injection, efficacy of cell delivery can be enhanced.

### Example 3

### Islet Grafts into Intramuscular Space

### A Preliminary Study Using a Biodegradable Scaffold Enriched with Vascular Growth Factors

Obstacles for successful islet transplantation are related to direct contact of islets with the blood stream and the liver as transplant site and include: IBMIR, high concentration of toxic immunosuppressive agents in the liver, and lack of noninvasive method to monitor islet function. To overcome those obstacles, the present example examines intramuscular islet implantation using a novel biocompatible scaffold which facilitates islet engraftment by creation of a new microenvironment and allows noninvasive monitoring of β-cell function by PET imaging.

### Material and Methods

Bioscaffold was manufactured from biodegradable alginate which contained VEGF and platelet derived growth factor (PDGF) with ability for gradual release. Additionally, it contained cyclic arginine-glycine-aspartic acid (RGD) peptide to increase extracellular signaling for both islets and endothelial cells by binding to αVβ3 and α5β1. Bioscaffold was implanted into rectus abdominal muscle 2 weeks before autologous islet transplantation in streptozotocin diabetic Lewis rats.

### Animals and Study Design

All animal studies were reviewed and approved by the Columbia University Institutional Animal Care and Use Committee. Male Lewis rats provided by Harlan Sprague Dawley (Indianapolis, Ind.) weighing between 200 and 250 g served as islet donors and transplant recipients in this study. Briefly, rats were divided into 5 groups of 12 rats each depending on the treatment they would receive. Two weeks prior to scheduled transplantation (transplant day −14), groups one and two underwent surgery for implantation of scaffolds and group three underwent a sham surgery described below. Four days prior to transplantation (day −4), all animals were rendered chemically diabetic. Finally on day 0, animals received transplant with syngeneic islets.

### Scaffold Preparation and Implantation

On day −14, animals in groups one, two, and three were anesthetized with isoflurane gas with concentrations ranging between 1-5%. Skin was opened in the midline and then dissected away from the underlying muscle. Muscle was then carefully dissected just lateral to the midline to identify the transversalis fascia ventral to the peritoneum. Blunt dissection was then used to create two pockets, one cephalad and one caudad in a plain between the transversalis fascia and the overlying abdominal musculature. Each pocket was made large enough to accommodate a scaffold 10 mm in diameter. Scaffolds were then implanted into those pockets in animals in groups one and two before closing the pockets and then skin in separate layers. Animals in group three were closed without scaffold implantation.

### Induction of Diabetes

On transplant day −4, all animals in groups 1-5 received injections of streptozotocin (STZ, Sigma Aldrich, St. Louis, Mo.) at a dose of 50 mg/kg via the penile vein under isoflurane anesthesia. Daily fasting blood glucose was measured with a Bayer Ascensia Elite XL glucometer to ensure diabetic status. Animals were considered diabetic if blood glucose values were greater than 300 mg/dL on each day.

### Islet Isolation

On transplant day 0, islets were harvested from Lewis donors. After intraperitoneal injection with ketamine (85 mg/kg) and xylazine (5 mg/kg) anesthesia, each donor's abdominal cavity was opened in the midline. Bowel and liver were retracted to expose the common bile duct, which was clamped at the ampulla of Vater. The inferior vena cava was ligated to exsanguinate the tissue. A 20-gauge needle inserted into the duct was then used to distend the pancreas with 12-15 mL of cold collagenase solution (1 mg/mL collagenase from Roche, Indianapolis, Ind.) dissolved in HBSS (Invitrogen, Carlsbad, Calif.). The pancreas was then excised and placed in a Petri dish in a water bath at 37 degrees C. for 10-20 minutes until adequate digestion had occurred. Collagenase was then washed out of pancreatic tissue before islets were separated from acinar tissue on a ficoll density gradient (purchased from Sigma Aldrich, St. Louis, Mo.). Ficoll concentrations of 24, 20, 16, and 12 were used and islets were extracted from the first two interfaces. Tissues from the two different interfaces were kept separate throughout the isolation process.

### Islet Yield and Viability

Islet yield was quantified by hand counting of 200 μL samples of dithizone-stained islet isolate (Diphenylthiocarbazone (dithizone) purchased from Sigma Aldrich, St. Louis, Mo.) under 20× magnification. Islet viability was assessed with double staining with SYTO 13/Ethidium bromide (EB) as described by Barnet et al. Twenty μL of 25 μM SYTO 13 and 20 μL of 25 μM EB were added to 450 μL of D-PBS. The mixture was then combined with 45 μL of islet isolate. Following several minutes of incubation, 50 islets were evaluated for percent viability.

### Insulin Stimulation Index

Retrospectively, islet quality was confirmed with insulin stimulation index according to a protocol adapted from that developed by Eirzirik et al. Briefly, 200 isolated, hand-picked islets were washed twice with low-glucose (1.7 mM) media. From those, 5 groups of 20 islets measuring 100 to 150 μM in diameter were placed in separate containers. Next, islets were sequentially pre-incubated with low glucose media, incubated with low-glucose media, and incubated with high-glucose media (16.7 mM). After each incubation, the media was removed from the islets and frozen for ELISA analysis. Following high-glucose incubation, islets were washed with PBS and added to acid ethanol before sonication and freezing for ELISA analysis.

### Islet Implantation

After isolation, 2400 islets were resuspended from final centrifugation pellets into volumes of approximately 0.4-0.5 mL of HBSS. High islet quality was confirmed before each injection by viability >90% and retrospectively by insulin stimulation index over 4. Study animals were anesthetized under isoflurane gas and opened in the midline. Skin was dissected away from the abdominal musculature and the peritoneum was opened such that both sides of the muscle could be visualized. For animals in groups 1 and 2, scaffolds were identified by palpation and islets were injected onto scaffolds in an intramuscular wheal via an 18-gauge needle. Islets from the first interface from the ficoll separation with purity around 90% were injected onto the cephalad part of the scaffold while islets from the second interface-purity 60% were loaded onto the caudad part. Animals from groups 3 and 4, which had not been pre-implanted with scaffolds, had islets injected intramuscularly in a similar fashion. Group 5, as a control, received no islets.

### Graft Monitoring: Metabolic Function, Histologic Examination

Transplanted animals were monitored with daily blood glucose measurements over the first two weeks post-transplant followed by bi-weekly measurements. Six-hour fasting measurements were obtained. In addition biweekly weight measurements were made.

Six animals from each of the treatment groups were sacrificed on transplant day 0 for histologic examination. Right abdominal muscle from the inferior costal margin to the pelvis was excised and preserved with formalin. Sections were taken through the scaffold for animals in groups 1 and 2 and through muscle for animals in groups 3, 4 and 5. H&E and factor VIII staining was carried out to examine vascularity of the tissue. Vessels within five separate high-power fields at 400× magnification were counted.

Insulin staining. From the remaining six animals in each group, tissue samples were taken in a similar fashion at two months post-transplantation. Those samples were additionally stained for insulin to demonstrate the presence of islets.

IPGGT. Intraperitoneal glucose tolerance testing (IPGTT) was also carried out on study rats at two months post-transplant. Glucose boluses of 1 g glucose/kg body weight were administered to unanesthetized animals, and blood glucose was measured at 0, 30, 60, 90, and 120 minutes post-injection. Area under the curve (AUC) for glucose excursions was calculated for comparison.

### Beta Cell Imaging

Additionally, at two week post-transplant, beta-cell imaging using a micro-PET scanner was performed. Following a protocol recently developed at Columbia University by Harris et al for imaging pancreatic beta cells, ¹¹C labeled dihydrotetrabenazine ([¹¹C]DTBZ) was administered via the penile vein at a dose of 1 μCi/g suspended in 0.4 mL saline. This ligand selectively binds vesicular monoamine transporter type 2 (VMAT-2), which is expressed in pancreatic beta cells, in the CNS and to a much smaller degree in other abdominal organ tissue.

### Results

### Restoration of Normoglycemia and Islet Implantation Success.

A goal of the islet transplantation is not only the survival of the islets in a new environment but also to resume function and restore normoglycemia in otherwise hyperglycemic and diabetic animals. Therefore, islet implantation success was defined as fasting glucose <100 mg/dL after the islet implantation on day +5 through +60. Islet implantation success rate was defined as the percentage of animals in each group with fasting blood glucose values in that range (N=6). Successful implantation was achieved in all animals (6/6) transplanted with islets into the fully enriched scaffold (group 1: gel+VEGF/PDGF), in 50% (3/6) of animals with the same scaffold but without VEGF or PDGF (group 2); and 33% (2/6) of animals without surgical pretreatment (group 4). All control animals (sham operated—group 3; or not transplanted—group 5) remained hyperglycemic (p<0.05). Mean glucose level oscillated around 100 mg/dL for animals from group 1 (gel+VEGF/PDGF), whereas in other groups it was statistically higher, p<0.05. As an additional control, removal of the scaffolds in normoglycemic animals from the gel+VEGF/PDGF group on day +60 led to prompt return of diabetes and hyperglycemia.

### Improvement in Metabolic Control Measured By Intraperitoneal Glucose Tolerance Test and Body Weight Increase.

The intraperitoneal glucose tolerance test is more challenging for islets and is a more sensitive gauge of their function. Results depend not only on the beta cells' quality, but also on their engraftment and vasculature development. Calculation of area under the curve (AUC) of serum glucose excursion during the test allows comparison of islets' functional capacity to restore normoglycemia, with smaller areas corresponding to better islet function. AUC was AUC was significantly lower for the gel+VEGF/PDGF group than for others (2-4 fold) confirming better islet engraftment in presence of scaffolds enriched with vascular grow factors. Gel+VEGF/PDGF group animals also gained weight significantly better than animals in the other groups, which confirms better function of the islets and overall better metabolic control in those animals (p<0.05).

### Development of Fibrovascular Tissue and Robust Vasculature at the Implantation “Bed.”

We hypothesized that success of islet implantation and their functional capacity depends on proper blood supply at the time of implantation and afterwards. Therefore, we evaluated the development of the vascular bed in the transplant site just before islet implantation in our experimental animals. Scaffolds were surgically removed and evaluated for vessel development 2 weeks after their implantation and just before islet injection would have occurred. Hemotoxylin and eosin staining showed tissue containing fibrovascular tissue penetrating scaffolds placed between muscular layers. Next, preserved tissue was stained for factor VIII, which is specific for endothelial cells and identifies blood vessels. The number of capillaries stained with factor VIII per high power field was significantly higher in the gel+VEGF/PDGF group compared to the other groups, 2-5 fold, p<0.05. Together with better metabolic function in animals from this group, this histologic examination confirms the significance of vasculature development within implantation “bed.”

### Confirmation of the Presence and Function of the Islets 2 Months After Implantation.

In order to confirm the role of the implanted islets in glucose control, on day 60 after transplantation, we removed the tissue from the implantation site. In euglycemic animals this caused prompt hyperglycemia and diabetes. Removed tissue stained for insulin is presented. Cells staining positively for insulin are seen within the scaffold, especially in proximity of vessels at the scaffold-muscle interface.

### Visualization of the Implanted Islets in PET.

As islet allografts can only be monitored by metabolic measures, which only detect graft dysfunction after substantial islet mass has already been lost, we tested a newly developed islet imaging technique. This method uses PET detection of a radiolabeled [¹¹C] dihydrotetrabenazine (DTBZ) molecule that acts as a ligand for vesicular monoamine transporter type 2 (VMAT2), which is heavily expressed by viable beta cells. This method, which has been shown to be effective for imaging islets in the pancreas, cannot be applied to islets infused into the liver because of the strength of the background signal. This technique has been never tested for an extrapancreatic location. In animals in the gel+VEGF/PDGF group, PET scan produced a strong signal within the right abdominal wall corresponding to the location of the transplanted islets. In contrast, there was no activity at the same site in hyperglycemic control animals with primary non-function. Since activity of the radiotracer allows for estimation of viable beta-cell mass in the native pancreas, our results indicate that this method has a great potential for assessment and monitoring of the transplanted islet function and mass as well. More importantly, a change in the signal precedes metabolic changes and allows for prompt local or systemic intervention preventing irreversible loss of transplanted islets.

### CONCLUSIONS

The use of a novel biocompatible scaffold containing RGD peptide and vascular growth factors significantly increases islet engraftment and extends their survival after intramuscular islet transplantation. Such approach may be attractive alternative for intraportal islet infusion, with great potential for preconditioning, immunological manipulation and increased effectiveness of islet transplantation with advantage of minimally invasive procedure.

### Example 4

### Visualization of Extrahepatic Rodent Islet Transplants with [C-11]DTBZ

Objectives: In the treatment of diabetes, islet transplantation (iTx) to the liver reestablishes normal feedback regulation of insulin secretion and normoglycemia. The Edmonton iTx protocol is associated with good short-term success but only a 10-15% success rate by 5 years post-iTx. Several mechanisms for iTx failure have been proposed including failure of initial engraftment, inflammatory responses, allo- or autoimmune response, and immunosuppressive drug-induced β-cell toxicity. Understanding islet graft failure and a non invasive method to estimate transplanted β-cell mass seems prerequisite before iTx outcomes improve. β-cell mass (BCM) measurements by PET with [¹¹C] DTBZ is not suitable for islets transplanted to the liver due to catabolism of the radioligand. Here we show feasibility of estimating BCM in iTx to an extrahepatic site—the intramuscular space of the abdominal wall prevascualrized with alginate scaffold containing RGD peptide and vascular growth factors. Methods. Normal Lewis rats were made diabetic with streptozotocin and then transplanted with 3000 purified allogeneic ACI islets. ITx reversed diabetes by day 2 and the abdomen was imaged (90 min) dynamically using 250+/−50 μCi [¹¹C]DTBZ and a Concorde microPET scanner on day six post Tx.

Results: In transplanted rodents without reversal of hyperglycemia and sham operated rodents, no preperitoneal uptake of radioligand was demonstrated. In diabetic rodents with euglycemia restored by iTx, an intramuscular islet cell mass is clearly revealed by uptake of [11C]DTBZ.

Conclusion: For islets transplanted to non hepatic sites, PET scans with [¹¹C]DTBZ may offer a means to monitor islet graft function and survival.

### Example 5

### Beta Cell Mass Imaging after Transplantation into an Extrahepatic Site

In the treatment of diabetes, cadaveric islet transplantation to the liver reestablishes normal feedback regulation of insulin secretion and long-term normoglycemia. The Edmonton transplantation protocol is associated with good short-term success but only a 10-15% success rate by 5 years post-transplantation. Several mechanisms for transplant failure have proposed including failure of initial engraftment, hepatic inflammatory responses, allo- or autoimmune response, and immunosuppressive drug-induced β-cell toxicity. Understanding islet graft failure and a non invasive method to estimate transplanted beta cell mass seems prerequisite before islet transplantation outcomes improve. The feasibility of in situ non invasive beta cell mass determinations by PET scans with [¹¹C] DTBZ has been demonstrated in rodent models of diabetes but is not suited to measuring beta cell mass in islets transplanted to the liver due to catabolism of the radioligand. Here we show feasibility of estimating beta cell mass in islets transplanted to an extrahepatic site, the intramuscular space of the abdominal wall. Normal Lewis rats were induced to stable diabetes with streptozotocin (More than three consecutive daily blood glucose measurements >250 mg/dl), and then transplanted with 3000 purified allogeneic ACI islets into prevascualrized intramuscular scaffold containing RGD peptide and slowly realizing vascular growth factors. Islet transplantation reversed diabetes by day 2 and the abdomen was imaged dynamically using 300 microcuries [¹¹C] DTBZ and a Concorde microPET scanner. In transplanted rodents who did not show reversal of hyperglycemia and sham operated rodents, no preperitoneal uptake of radioligand could be demonstrated.

### REFERENCES

1. Chen et al., Spatio-temporal VEGF and PDGF Delivery Patterns Blood Vessel Formation and Maturation. Pharm. Res. 24(2):258-64 (2007).
2. Christman and Lee, Biomaterials for the treatment of myocardial infarction. J. Am. Coll. Cardiol. 48:907-13 (2006).
3. Christman et al., Injectable fibrin scaffold improves cell transplant survival, reduces infarct expansion, and induces neovasculature formation in ischemic myocardium. J. Am. Coll. Cardiol. 44(3):654-60 (2004).
4. Dusseault et al., Evaluation of alginate purification methods: effect on polyphenol, endotoxin, and protein contamination. J. Biomed. Mater. Res. 76:243-51 (2006).
5. Hill et al., Regulating activation of transplanted cells controls tissue regeneration. Proc. Natl. Acad. Sci. USA. 103(8):2494-9 (2006a).
6. Hill et al., Designing scaffolds to enhance transplanted myoblast survival and migration. Tissue Eng. 12(5):1295-304 (2006b).
7. Kano et al., VEGF-A and FGF-2 synergistically promote neoangiogenesis through enhancement of endogenous PDGF-BPDGFRbeta signaling. J Cell Sci. 118(Pt 16):3759-68 (2005).
8. Klock et al., Biocompatibility of mannuronic acid-rich alginates. Biomaterials 18(10):707-13 (1997).
9. Kutschka et al., Collagen matrices enhance survival of transplanted cardiomyoblasts and contribute to functional improvement of ischemic rat hearts. Circulation 114(1 Suppl):I167-73 (2006).
10. Leor et al., Bioengineered cardiac grafts: A new approach to repair the infarcted myocardium? Circulation 102(19 Suppl 3):56-61 (2000).
11. Lunde et al., Intracoronary injection of mononuclear bone marrow cells in acute myocardial infarction. N. Engl. J. Med. 355(12):1199-209 (2006).
12. Markusen et al., Behavior of adult human mesenchymal stem cells entrapped in alginate-GRGDY beads. Tissue Eng. 12(4):821-30 (2006).
13. Martens et al., Mesenchymal lineage precursor cells induce vascular network formation in ischemic myocardium. Nat. Clin. Pract. Cardiovasc. Med. 3 Suppl 1:S18-22 (2006).
14. Miyahara et al., Monolayered mesenchymal stem cells repair scarred myocardium after myocardial infarction. Nat. Med. 12(4):459-65 (2006).
15. Nuttelman et al., Synthetic hydrogel niches that promote hMSC viability. Matrix Biol. 24(3):208-18 (2005).
16. Rowley et al., Alginate hydrogels as synthetic extracellular matrix materials. Biomaterials 20(1):45-53 (1999).
17. Schachinger et al., Intracoronary bone marrow-derived progenitor cells in acute myocardial infarction. N. Engl. J. Med. 355(12):1210-21 (2006).
18. Teng et al., Massive mechanical loss of microspheres with direct intramyocardial injection in the beating heart: implications for cellular cardiomyoplasty. J. Thorac. Cardiovasc. Surg. 132(3):628-32 (2006).
19. Zimmermann et al., A novel class of amitogenic alginate microcapsules for long-term immunoisolated transplantation. Ann. N.Y. Acad. Sci. 944:199-215 (2001).
20. Zimmermann et al., Engineered heart tissue grafts improve systolic and diastolic function in infarcted rat hearts. Nat. Med. 12(4):452-8 (2006).

What is claimed is:

1. A porous three dimensional scaffold comprising purified alginate molecules that are conjugated to cyclic RGD peptides.

2. The porous three dimensional scaffold of claim 1, wherein the alginate molecules are poly-mannuronic or poly-guluronic acid molecules.

3. The porous three dimensional scaffold of claim 1, wherein the cyclic RGD peptides comprise a sequence RGDxy, wherein “x” is phenylalanine or tyrosine, and “y” is cysteine, glutamic acid, lysine or valine.

4. The porous three dimensional scaffold of claim 1, wherein the alginate molecules are purified to contain less than 0.305% protein.

5. The porous three dimensional scaffold of claim 1, wherein the alginate molecules are purified to contain less than 12.5 EU endotoxin per gram dry alginate.

6. The porous three dimensional scaffold of claim 1, further comprising one or more components selected from the group consisting of cells, immunomodulatory factors, and growth factors.

7. The porous three dimensional scaffold of claim 6, wherein the cells are stem cells, myocytes, human bone marrow derived mesenchymal precursor cells, or islet cells.

8. The porous three dimensional scaffold of claim 6, wherein the growth factors are PDGF, VEGF, or thymosin beta 4.

9. The porous three dimensional scaffold of claim 6, wherein the immunomodulatory factors are antibodies, synthetic drug or peptide.

10. The porous three dimensional scaffold of claim 1, wherein the alginate molecules are purified by a method comprising the steps of dissolving the alginate molecules in an acidic buffer; removing protein, DNA, RNA and endotoxin by neutral and active charcoal treatment, and purifying by filtration through bioactive filter membranes and precipitation with ethanol.

11. A composition comprising the porous three dimensional scaffold of claim 1.

12. A method of promoting tissue or cell transplantation, comprising the steps of:
i. preparing a porous three dimensional scaffold of claim 1;
ii. loading the porous three dimensional scaffold with cells or tissue; and
iii. transplanting the loaded porous three dimensional scaffold into a human or animal, thereby obtaining better transplantation results as compared to transplantation without the porous three dimensional scaffold.

13. The method of claim 12, wherein the cells are stem cells, myocytes, human bone marrow derived mesenchymal precursor cells, or islet cells.

14. The method of claim 12, wherein the porous three dimensional scaffold further comprises one or more immunomodulatory factors or growth factors.

15. A method of promoting tissue or cell transplantation, comprising the steps of:
i. creating a vascular bed by transplanting a porous three dimensional scaffold of claim 1 into a human or animal; and
ii. transplanting cells or tissues into the vascular bed, thereby obtaining better transplantation results as compared to transplantation without using the porous three dimensional scaffold.

16. The method of claim 15, wherein the cells are stem cells, myocytes, human bone marrow derived mesenchymal precursor cells, or islet cells.

17. The method of claim 15, wherein the porous three dimensional scaffold further comprises one or more immunomodulatory factors or growth factors.

18. A method of promoting cell transplantation to heart, comprising the steps of:
i. preparing a porous three dimensional scaffold of claim 1;
ii. loading the porous three dimensional scaffold with stem cells or myocytes; and
iii. transplanting the loaded porous three dimensional scaffold into a heart, thereby obtaining better transplantation results as compared to transplantation without the porous three dimensional scaffold.

19. The method of claim 18, wherein the porous three dimensional scaffold further comprises one or more immunomodulatory factors or growth factors.

20. The method of claim 19, wherein the immunomodulatory factor or growth factor is PDGF, VEGF, or thymosin beta 4.
